(19) United States
(12) Patent Application Publication
(10) Pub. No.: US 2026/0275249 A1
(43) Pub. Date: Sep. 17, 2026

(54) Bleach Catalyst With Improved Performance, Granule Comprising The Bleach Catalyst, And Composition Comprising Said Catalyst Or Said Granule

(71) Applicant: Reckitt Benckiser Vanish B.V., Hoofddorp (NL)

(72) Inventors: Andre Chieffi, Milano (IT); Torsten Roth, Mannheim (DE); Federico Spolaore, Milano (IT); Diego Simionato, Milano (IT)

(73) Assignee: Reckitt Benckiser Vanish B.V., Hoofddorp (NL)

(21) Appl. No.: 19/168,144

(22) PCT Filed: Apr. 2, 2024
(86) PCT No.: PCT/EP2024/058915

§ 371 Date: Sep. 23, 2025

(30) Foreign Application Priority Data
Mar. 30, 2023 (GB) 2304721.0

(51) Int. Cl. C11D 3/39; C07D 275/06; C07D 417/04; C11D 3/00; C11D 3/32; C11D 3/34; C11D 11/00

(52) U.S. Cl. CPC C11D 3/392; C07D 275/06; C07D 417/04; C11D 3/0068; C11D 3/32; C11D 3/349; C11D 3/3942; C11D 11/0088

(57) ABSTRACT

The present invention relates to a bleach catalyst, to a granule comprising the bleach catalyst and a carrier, and to a cleaning composition comprising the bleach catalyst and/or the granule, at least one bleaching agent and at least one bleach activator. The invention also relates to the use of the cleaning composition for the removal of stains and/or the removal of malodour from a garment. It also relates to a method of washing clothes, which comprises providing the composition according to the invention to a laundry washing machine and performing the washing at a temperature lower than 60° C. and for a time of less than 120 minutes.

The present invention relates to a bleach catalyst, to a granule comprising the catalyst, to a cleaning composition comprising said catalyst or said granule, to a method of preparing the granule, to the use of the composition for the removal of stains and/or for the removal of malodour from a garment, and to a method of washing clothes.

### BACKGROUND

Bleach catalysts are commonly used to catalyse the oxidation of oxidable compounds on, for example, surfaces such as fabrics, dishes, toilets, countertops, and the like. For example, bleach catalysts are used to catalyse the bleaching action of hydrogen peroxide or of compounds that release hydrogen peroxide, such as peroxy compounds, against various stains.

In the 20^{th} century metal-containing catalysts containing macrocyclic ligands were described for use in bleaching compositions. However, these metal-containing bleach catalysts, in particular manganese-containing catalysts are known to damage textile fabric and have the ability to locally stain or discolour substrates. An alternative to this type of metal-containing catalysts was proposed in Patent Application WO98/39098, which describes catalytic systems and methods for oxidising materials in the presence of catalysts which are complexes of transition metals such as Mn, Fe, or Cr, with selected marcopolycyclic rigid ligands, such a cross-bridged macropolycyclic ligands.

Another type of catalysts was proposed in European Patent Application EP0453003, in which it is described that certain types of sulfonimines can be used as bleach catalysts. A particularly preferred sulfonimine described in this document is 3-methyl-1,2-benzisothiazole-1,1-dioxide, which is a cyclic sulfonimine.

However, different types of cyclic sulfonimines have different catalytic properties. A bleach catalyst, when used in laundry processes, is used to catalytically accelerate the oxidation of stains with systems comprising a source of oxygen (e.g. H₂O₂) and a bleach activator. The catalytic cycle involves the following steps: i) hydrogen peroxide perhydrolyses the bleach activator (e.g., TAED) to produce peracetic acid, ii) the peracetic acid oxidises the sulfonimine-type bleach catalyst to generate an oxaziridine, iii) the oxaziridine reacts with the stain in a much faster manner than the peracid, iv) regeneration of the sulfonimine-type bleach catalyst. This cycle is quickly repeated several times, which leads to a much faster, i.e. better kinetics, bleaching of the stain.

When considering laundry items, consumers associate desirability and wearability of a garment with characteristics such as e.g., whiteness, freshness, and/or absence of stains. It is preferred when these characteristics are achieved in a simple way.

Stains containing polyphenols are particularly difficult to remove with common laundry detergents, chlorine bleaches, existing laundry additives or any home remedies. Example of polyphenol-containing foods are tea, red wine, curry, and chocolate. These compounds have been used as dyes and for tanning garments, which gives an idea of how complicated it is to remove polyphenol-containing stains from a garment.

Curcumin is a hydrophobic polyphenol derived from the rhizome of turmeric (Curcuma longa). Turmeric is a strong natural colorant and the additional presence of oil, fat, meat and other protein components like egg and milk make this type of stains extremely complicated to clean. This means that in cuisines where curry/turmeric plays an important role, people are left with no solution and they have no choice than to cope with curry/turmeric stains residues on their garments, even after washing.

In this sense, normally, people are required to follow long cleaning procedures, involving pre-treatment, vigorous rubbing action, or washings at high temperatures to achieve acceptable stain and malodour removal. For example, washing operations are normally performed at temperatures between 40° C. and 60° C. A topic of particular interest nowadays is the need of washing at lower temperatures, e.g. at 40° C. or lower temperatures, where peroxy compounds need to be activated by bleach activators and/or bleach catalyst. These peroxy compounds are for example, perborates, persulfates or percarbonates, which are added to detergents and cleaning agents to obtain a satisfactory bleaching effect during cleaning.

Hence, there is a need of a bleach catalysts with improved catalytic properties. In addition, there is a need of a bleach catalyst, which allows for the removal of stains, in particular stains comprising a polyphenolic compound such as turmeric and/or curry stains.

Therefore, it is an object of the present invention to provide a bleach catalyst with improved catalytic properties.

It will be recognised that when bleach catalysts are added to detergents and cleaning agents, it can be difficult to ensure a homogeneous distribution of small quantities of the catalyst. Therefore, bleach catalysts can be provided in the form of granules. This not only ensures a homogeneous incorporation of small quantities of these ingredients into a composition, but also increases their storage stability.

A further object of the present invention is then to provide a granule comprising a bleach catalyst with improved catalytic properties, wherein the granule exhibits good storage stability and is easy to be homogenously added to a cleaning composition.

Another object of the present invention is to provide a method of preparing a granule comprising a bleach catalyst with improved catalytic properties.

It is a further object of the present invention to provide a cleaning composition, which can be used to clean hard and/or soft surfaces, in particular soft surfaces such as textiles. The composition is in particular effective at removing stains, such as polyphenol-containing stains (e.g. turmeric and/or curry stains).

It is a further object of the present invention to provide a method of washing clothes at temperatures lower than 60° C. and preferably at short washing cycles, for example, less than 120 minutes.

### SUMMARY OF THE INVENTION

In a first aspect, the present invention relates to a bleach catalyst having formula (1)

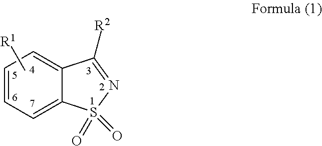

wherein:
   R¹ is H, halogen, hydroxy, amino, nitro, cyano, carboxy, alkyl, C₁-C₁₀-alkoxy, C₁-C₁₀-alkylamino, phenyl, substituted phenyl, or a heterocyclic ring;
   R² is an organic group comprising a carbon atom that is bonded to the third carbon of the bleach catalyst,
   the partial charge density of the C═N bond of the bleach catalyst is less than −0.180, and
   the ClogP of the bleach catalyst is lower than 2.

In an embodiment, the ClogP of the bleach catalyst is lower than 1.8, preferably lower than 1.5, more preferably lower than 1, even more preferably between 0.2 and 0.90, most preferably between 0.3 and 0.85.

In another embodiment, the partial charge density of the C═N bond is less than −0.190, preferably less than −0.195, more preferably less than −0.200.

In a preferred embodiment, R¹ is H, hydroxy, or C₁-C₁₀-alkoxy; preferably H.

In an embodiment, R² is alkyl, heteroalkyl, alkenyl, alkynyl, aryl, substituted aryl, aryl with multiple or fused rings in which at least one ring is aromatic, heteroaryl, or heterocyclic ring.

In another preferred embodiment, R² is an organic group in which the carbon atom that is bonded to the third carbon of the bleach catalyst is not bonded to any hydrogen atom.

In an embodiment R² is:
a branched alkyl or heteroalkyl having 4 to 16 carbon atoms, preferably 4 to 12 carbon atoms, more preferably 4 to 10 carbon atoms, even more preferably 4 to 8 carbon atoms;
a cycloalkyl-, cycloalkenyl-, cycloalkynyl-, or aryl-substituted linear or branched alkyl having 2 to 16 carbon atoms in the alkyl chain, preferably having 2 to 12 carbon atoms in the alkyl chain, more preferably having 2 to 10 carbon atoms in the alkyl chain, even more preferably 2 to 8 carbon atoms in the alkyl chain;
a branched or cyclic alkenyl having 3 to 16 carbon atoms, preferably having 3 to 12 carbon atoms, more preferably having 3 to 10 carbon atoms, even more preferably having 3 to 8 carbon atoms;
a heterocyclic alkenyl;
a cycloalkyl-, cycloalkenyl-, cycloalkynyl-, or aryl-substituted linear or branched alkenyl having 2 to 16 carbon atoms in the alkenyl chain, preferably having 2 to 12 carbon atoms in the alkenyl chain, more preferably having 2 to 10 carbon atoms in the alkenyl chain, even more preferably having 2 to 8 carbon atoms in the alkenyl chain;
a linear alkynyl having 2 to 16 carbon atoms, preferably having 2 to 12 carbon atoms, more preferably having 2 to 10 carbon atoms, even more preferably having 2 to 8 carbon atoms;
a branched alkynyl having 5 to 16 carbon atoms, preferably having 5 to 14 carbon atoms, more preferably having 5 to 12 carbon atoms, even more preferably having 5 to 10 carbon atoms;
a cycloalkyl-, cycloalkenyl-, cycloalkynyl-, or aryl-substituted linear or branched alkynyl having 2 to 16 carbon atoms in the alkynyl chain, preferably having 2 to 12 carbon atoms in the alkynyl chain, more preferably having 2 to 10 carbon atoms in the alkynyl chain, even more preferably having 2 to 8 carbon atoms in the alkynyl chain;
a substituted aryl,
an aryl with multiple or fused rings in which at least one ring is aromatic, or
a heteroaryl.

In a preferred embodiment, R² is:
a branched alkyl or heteroalkyl having 4 to 16 carbon atoms, preferably 4 to 12 carbon atoms, more preferably 4 to 10 carbon atoms, even more preferably 4 to 8 carbon atoms, or
a cycloalkyl-, cycloalkenyl-, cycloalkynyl-, or aryl-substituted linear or branched alkyl having 2 to 16 carbon atoms in the alkyl chain, preferably having 2 to 12 carbon atoms in the alkyl chain, more preferably having 2 to 10 carbon atoms in the alkyl chain, even more preferably 2 to 8 carbon atoms in the alkyl chain;
then the carbon in R² that is bonded to the third carbon atom of the bleach catalyst having formula (1) is a quaternary carbon atom.

In another embodiment, R² is:
a heterocyclic alkenyl,
a substituted aryl,
an aryl with multiple or fused rings in which at least one ring is aromatic, or
a heteroaryl.

In another embodiment, R² is a heterocyclic alkenyl or a heteroaryl.

In an embodiment, R² is pyridyl, quinolyl, isoquinolinyl, acridinyl, pyrazinyl, quinoxalinyl, imidazolyl, benzimidazolyl, purinyl, pyrazolyl, indazolyl, pyrimidinyl, quinazolinyl, pyridazinyl, or cinnolinyl.

In a specially preferred embodiment, R² is a pyridyl, preferably 2-pyridyl or 3-pyridyl.

In a second aspect, the invention relates to a granule comprising the bleach catalyst according to the first aspect of the invention and a carrier.

In an embodiment, the carrier is selected from the group consisting of sulphates, carbonates, sesquicarbonates, silicates, aluminosilicates, zeolites, precipitated silicas, fumed silicas, and combinations thereof; preferably selected from the group consisting of sulphates, carbonates, sesquicarbonates, and combinations thereof; more preferably selected from the group consisting of the alkali metal salts of sulphates, carbonates, sesquicarbonates, and combinations thereof.

In another embodiment, the granule comprises between 0.1 wt. % and 40 wt. % of the bleach catalyst, based on the total weight of the granule, preferably between 0.2 wt. % and 10 wt. %, even more preferably between 0.5 wt. % and 5 wt. %.

In a preferred embodiment, the granule further comprises at least one surfactant, preferably a non-ionic surfactant.

In another embodiment, the granule further comprises a binder.

In a preferred embodiment, the granule has a particle size of between 200 μm and 2000 μm, preferably between 300 μm and 1500 μm, more preferably between 500 μm and 1200 μm.

In a third aspect, the invention relates to a method of preparing the granule according to the second aspect of the invention, wherein the method comprises the steps of:
a) Preparing a solution by dissolving the bleach catalyst in a solvent system,
b) Adding the solution prepared in step a) to the carrier,
c) Allowing the solvent system to evaporate partially or completely,
   wherein the solvent system has a dielectric constant below 80, a vapour pressure above 5 kPa and a boiling point below 100° C.

In an embodiment, the solvent system comprises one or more solvents selected from the group consisting of water, methanol, ethanol, propylene carbonate, acetone, acetonylacetone, diacetone alcohol, ethyl acetate, 2-propanol, ethylene glycol, propylene glycol, glycerine, diethylene glycol, dipropylene glycol monomethyl ether and dimethylformamide.

In a preferred embodiment, the solvent system further comprises at least one surfactant, preferably a non-ionic surfactant.

In a fourth aspect, the invention relates to a cleaning composition comprising the bleach catalyst according to the first aspect of the invention and/or the granule according to the second aspect of the invention, at least one bleaching agent and at least one bleach activator.

In an embodiment, the at least one bleaching agent and/or the at least one bleach activator is/are partially or totally contained in the granule.

In an embodiment, the composition is a hard-surface cleaning composition or a soft-surface cleaning composition, preferably an automatic dishwashing composition or a laundry composition, more preferably a laundry detergent composition or a laundry additive composition, most preferably a laundry additive composition.

In another embodiment, the composition comprises between 0.001 wt. % and 10 wt. % of the bleach catalyst, based on the total weight of the composition, preferably between 0.005 wt. % and 5 wt. %, more preferably between 0.01 wt. % and 1 wt. %, most preferably between 0.025 wt. % and 0.5 wt. %, preferably wherein the bleach catalyst is partially or totally contained in the granule.

In a preferred embodiment, the at least one bleaching agent comprises at least one source of active oxygen selected from the group consisting of an inorganic peroxide, an organic peracid, hydrogen peroxide, and combinations thereof; preferably selected from the group consisting of sodium percarbonate, sodium perborate, ε-phthalimidoperoxy-hexanoic acid (PAP), peracetic acid, potassium peroxymonosulfate (KMPS), and combinations thereof; most preferably wherein said at least one bleaching agent is sodium percarbonate, preferably wherein the at least one bleaching agent is partially or totally contained in the granule.

In an embodiment, the at least one bleach activator is selected from the group consisting of tetraacetylethylenediamine (TAED), acetylated triazine derivatives, acetylated glycoluriles, acylimides, acetylated glucose, acetylated glycerol, acetylated phenolsulfonates, acetylated phenol carbonic acids, sodium nonanoyloxybenzenesulfonate (NOBS), 4-decanoyloxybenzoic Acid (DOBA), carbonic acid anhydrides, acetylated sugar derivatives, N-acetylated lactams, and combinations thereof; preferably said at least one bleach activator is tetra acetylethylenediamine (TAED), preferably wherein the at least one bleach activator is partially or totally contained in the granule.

In a fifth aspect, the invention relates to a use of the cleaning composition according to the fourth aspect of the invention for the removal of stains, preferably stains comprising a polyphenolic compound, more preferably wherein the stains comprise turmeric and/or curry stains and/or for the removal of malodour from a garment.

In an embodiment, for the removal of stains from a garment, wherein the garment is made of cotton, synthetic materials or a combination thereof, preferably wherein said synthetic material is polyester.

In a sixth aspect, the invention relates to a method of washing clothes, wherein the method comprises providing the composition according to the fourth aspect of the invention to a laundry washing machine and performing the washing:
at a temperature lower than 60° C., preferably lower than 40° C., more preferably lower than 30° C., even more preferably lower than 25° C.; and
for a time of less than 120 minutes, preferably less than 60 minutes, more preferably less than 45 minutes, even more preferably less than 40 minutes, most preferably less than 30 minutes.

### Definitions

The following definitions are used in the present application.

“alkyl” as used in the present application means a saturated aliphatic radical, such as straight or branched chain group having 1 to 16 carbon atoms, cycloalkyl groups having 3 to 16 carbon groups in their ring, alkyl substituted cycloalkyl groups, and cycloalkyl substituted alkyl groups.

Non-limiting examples of straight and branched chain groups are methyl, ethyl, butyl, isopropyl, and 2-ethylhexyl.

“heteroalkyl” as used in the present application means an alkyl group in which one or more atoms is a heteroatom, e.g., oxygen, nitrogen, sulphur, etc. Non-limiting examples of heteroalkyl groups are alkoxy, alkyl-substituted amino, and piperidinyl.

“aryl” as used in the present application means aromatic hydrocarbon radical, which can be substituted, have a single ring, multiple rings or fused rings in which at least one is aromatic. A non-limiting example of an aromatic single ring is phenyl. A non-limiting example of multiple rings is biphenyl. A non-limiting example of multiple fused rings in which at least one is aromatic is phenantryl.

When talking about an aryl with multiple rings or fused rings in which at least one is aromatic, it will be understood that these are polycyclic aryls.

“heteroaryl” as used in the present application means an aryl group in which one or more atoms is a heteroatom, e.g., oxygen, nitrogen, sulphur, etc. Non-limiting examples of heteroaryl groups are pyrrolyl, furanyl, and thiophenyl.

“alkaryl” as used in the present application means an aryl group with a saturated straight or branched chain having 1 to 16 carbon atoms; for example, ethylphenyl and decylphenyl.

“alkenyl” as used in the present application means any radical having at least one ethylenic double bond, including straight or branched chain groups having 2 to 16 carbon atoms and at least one double bond, cycloaliphatic alkenyl groups, and aryl substituted alkenyl groups. Non-limiting examples of alkenyl groups are octenyl and methyloctenyl.

“cycloalkenyl” as used in the present application means cyclic species with at least one ethylenic double bond in the ring. The ring has between 3 to 10 atoms. These species are also known as cyclic olefins. Non-limiting examples of cycloalkenyl or cyclic olefins are dicyclopentadiene, bicyclo compounds, and oxabicyclo compounds.

“alkynyl” as used in the present application means any radical having at least one triple bond, including straight or branched chain groups having 2 to 16 carbon atoms and at least one triple bond, cycloaliphatic alkynyl groups, and aryl substituted alkynyl groups. Non-limiting examples of alkenyl groups is butynyl.

“cycloalkynyl” as used in the present application means cyclic species with at least one triple bond in the ring. The ring can have between 5 to 10 atoms.

“halogen” as used in the present application means F, Cl, Br, or I.

“organic group” as used in the present patent application means a chemical group or radical that contains carbon-hydrogen or carbon-carbon bonds.

“pyridyl” as used in the present application means any of the three isomeric radicals derived from pyridine.

“quinolyl” as used in the present application means a radical from quinoline.

“isoquinolinyl” as used in the present application means a radical derived from isoquinoline.

“acridinyl” as used in the present application means a radical derived from acridine.

“pyrazinyl” as used in the present application means a radical derived from pyrazine.

“quinoxalinyl” as used in the present application means a radical derived from quinoxaline.

“imidazolyl” as used in the present application means a radical derived from imidazole.

“benzimidazolyl” as used in the present application means a radical derived from benzimidazole.

“purinyl” as used in the present application means a radical derived from purine.

“pyrazolyl” as used in the present application means a radical derived from pyrazole.

“indazolyl” as used in the present application means a radical derived from indazole.

“pyrimidinyl” as used in the present application means a radical derived from pyrimidine.

“quinazolinyl” as used in the present application means a radical derived from Quinazoline.

“pyridazinyl” as used in the present application means a radical derived from pyridazine.

“cinnolinyl” as used in the present application means a radical derived from cinnoline.

“ClogP” as used in the present application means logarithm of the partition coefficient of a compound defined as the ratio of concentrations of a compound in a mixture of two immiscible solvents at equilibrium, here n-octanol and water; i.e ClogP=log₁₀(partition coefficient); wherein partition coefficient

$$( P ) = \frac{{[ compound ]}_{n - octanol}}{{[ compound ]}_{water}} .$$

It is calculated using ChemDraw 12.0 (ChemOffice suite by Perkin Elmer), which uses a dataset from full compounds, or fragments, which are experimentally determined, and then modelled in small fragments. Fragment contributions are then added up, with correction factors.

“Partial charge density of the C═N bond” as used in the present application means the calculation of the electronegativity of the C═N bond by adding the calculated partial charge of both atoms C and N; in other words, the calculated partial charge of the bond between the third carbon atom and the nitrogen in the second position of the bleach catalyst having formula (1). Partial charge is more commonly known as atomic charges. This value represents the resulting distribution of the charges affected by the different electronegativity of atoms in the vicinity of the atom of interest; when an electrically neutral atom is bonded to another neutral atom that is more electronegative, its electrons are partially drown away. This leaves the region around that atom's nucleus with a partial positive charge, and it creates a partial negative charge on the atom to which the first is bonded.

In the present application the partial charge density of the C═N bond of all example molecules described in this document is calculated by using the desktop modelling program Chem3D suite in ChemDraw 12.0 (ChemOffice by Perkin Elmer). For an appropriate calculation of the minimisation energy of the of the bleach catalyst of interest, the molecules are drawn in ChemDraw 12.0. Subsequently the chemical structure must be cleaned up with the appropriate function and, in the Chem3D suite, the minimisation energy is reached using the MM2 force field computations tool of the program. After the minimum spatial energy is found, the partial charge density for all the atoms of the molecule, specifically those of the C (third carbon atom) and the N (nitrogen in the second position of the bleach catalyst of formula (1)) of the imine bond is computed with the Hückel charge density function of the program. The sum of the partial charge density of the two atoms (C and N) is used in this document to describe the partial charge density of the C═N imine bond.

“Laundry detergent composition” as used in the present application means a composition used primarily for the cleaning of garments and whose main action is the removal of grime and dirt.

“Laundry additive composition” as used in the present application means a composition used for providing a specific consumer benefit, such as: removal of stains, elimination of malodour, improvement of whiteness and/or disinfection of garments. This means that the mean action of a laundry additive is not the removal or grime and dirt. Furthermore, a laundry additive is commonly used as a pre-treatment, in soaking conditions or together with a laundry detergent composition.”

“Dielectric constant (E)” as used in the present application refers to the physico-chemical property measured at ambient temperature as reported in the Handbook of chemistry and physics, 88^{th} ed., CRC Press, Boca Raton, 2008. The dielectric constant of a solvent is a measure of its polarity. The higher the dielectric constant of a solvent, the more polar the solvent is. For example, the dielectric constant of water (80.1) is higher than that of methanol (33.0), therefore water is more polar than methanol.

“Vapour pressure (Vp)” as used in the present application refers to the physico-chemical property measured at 25° C. at 1 kPa (7.5 mmHg) as reported in the Handbook of chemistry and physics, 88^{th} ed., CRC Press, Boca Raton, 2008. The vapor pressure is the pressure exerted by a vapor in thermodynamic equilibrium with its condensed phases (solid or liquid) at a given temperature in a closed system. The vapor pressure is therefore an indication of a liquid's thermodynamic tendency to evaporate. For example, the vapor pressure of water (3.17) is lower than that of methanol (16.9), therefore water is less prone to evaporate.

“Boiling point (Bp)” as used in the present application refers to the normal boiling point measured in ° C. and reported as in the Handbook of chemistry and physics, 88^{th} ed., CRC Press, Boca Raton, 2008.

“Anhydrous” as used in the present application is to be understood as a composition, component or ingredient that has no more than 10 wt. % water, preferably no more than 5 wt. % water.

A list of physico-chemical properties (E, Vp, and Bp) for a set of solvents mentioned or referred to in the present patent application is shown in Table 1:

TABLE 1
Physico-chemical properties of selected solvents

| | ε/a.u. | Vp/kPa | Bp/° C. |
|---|---|---|---|
| Acetone | 21.01 | 30.8 | 56.05 |
| Acetonitrile | 36.64 | 11.90 | 81.65 |
| Benzyl alcohol | 11.92 | 0.015 | 205.31 |
| Ethanol | 25.30 | 7.87 | 78.29 |
| Ethyl acetate | 6.08 | 12.6 | 77.11 |
| Ethylene glycol | 41.4 | 0.01 | 197.3 |
| Tetrahydrofuran | 7.52 | 21.6 | 65.00 |
| Hexane | 1.89 | 20.2 | 68.73 |
| Toluene | 2.38 | 3.79 | 110.00 |
| Water | 80.10 | 3.17 | 100.00 |

### DETAILED DESCRIPTION OF THE INVENTION

In a first aspect, the present invention relates to a bleach catalyst having formula (1):

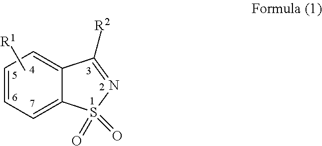

wherein:
   R¹ is H, halogen, hydroxy, amino, nitro, cyano, carboxy, alkyl, C₁-C₁₀-alkoxy, C₁-C₁₀-alkylamino, phenyl, substituted phenyl, or a heterocyclic ring;
   R² is an organic group comprising a carbon atom that is bonded to the third carbon of the bleach catalyst,
   the partial charge density of the C═N bond of the bleach catalyst is less than −0.180, and
   the ClogP of the bleach catalyst is lower than 2.

It will be understood that the partial charge density of the C═N bond of the bleach catalyst is given by the sum of the partial charge density of the bond between the third carbon atom and the nitrogen in the second position of the bleach catalyst of formula (1).

The present inventors have observed that the catalytic activity of the bleach catalyst of Formula (1) is highly dependent on two parameters: i) the hydrophobicity of the molecule, which is given by the ClogP, and ii) the electronegativity of the C═N bond, which is given by the partial charge density of the C═N bond.

The hydrophobicity of the bleach catalyst is related to its ability to dissolve in a typical aqueous environment, and this is given by the partition coefficient of the catalyst, i.e., ClogP. The present inventors have observed that it is essential, form a performance perspective, that the bleach catalyst has a ClogP lower than 2.

In addition, the electronegativity of the C═N bond is related to the catalytic cycle of the bleach catalyst, which goes through the formation of an oxaziridine (i.e. oxidation of the C═N bond).

Therefore, the electronegativity of this bond plays a paramount role in the catalytic activity of the molecule. Electronegativity can be described as the partial charge density in the C═N bond and is calculated by adding the partial charge of both atoms, N and C. The present inventors have observed that it is also essential, from a performance perspective, that the partial charge density of the C═N bond is less than −0.180. The person skilled in the art will recognise that in the bleach catalyst having formula (1) this parameter is influenced by R¹ and R².

Hence, the present inventors have observed that the catalytic activity of the bleach catalyst of Formula (1) is improved when the ClogP is lower than 2 and the R¹ and R² are such that the partial charge density of the C═N bond is less than −0.180.

In a preferred embodiment, the ClogP of the bleach catalyst is lower than 1.8, preferably lower than 1.5, more preferably lower than 1, even more preferably between 0.2 and 0.90, most preferably between 0.3 and 0.85.

In another preferred embodiment, the partial charge density of the C═N bond is less than −0.190, preferably less than −0.195, more preferably less than −0.200.

In an embodiment R¹ is H, hydroxy, or C₁-C₁₀-alkoxy; preferably H.

In a preferred embodiment, R² is alkyl, heteroalkyl, alkenyl, alkynyl, aryl, substituted aryl, aryl with multiple or fused rings in which at least one ring is aromatic, heteroaryl, or heterocyclic ring.

Some of the bleach catalyst according to the invention may be prone to degradation under storage conditions, which can be evidenced by colouration of the catalyst. This problem can be solved by adding a physical barrier to the catalyst, such as for example, a coating, or by making a granule comprising the bleach catalyst and ingredients that reduces or prevents such degradation. For a cyclic sulfonimine such as 3-methyl-1,2-benzisothiazole-1,1-dioxide, Patent Application US2018/0187130 to Weylchem Wiesbaden GmbH addresses the problem of degradation by providing a granulate of the sulfonimines with acids in solid form.

Other possible solutions to the degradation problem include, for example:
Wax-encapsulated particles having a core of particles including the bleach catalyst and further particles selected from the group of an organic peroxyacid, a diacyl peroxide, an inorganic peroxygen compound, a peroxygen bleach precursor and mixtures thereof. A description of this type of technology can be found in U.S. Pat. No. 5,480,577.
Solid compositions comprising the bleach catalyst and a C₆-C₁₈ fatty acid binding system for improving shelf stability of an activated bleach composition containing a peroxygen source and a bleach activating agent. A description of this type of technology can be found in US2019382692.
A composition comprising a core and a coating, wherein the bleach catalyst is present in the core. The coating being made of a water-soluble material that at least partially surrounds the core. The coating process comprises the steps of introducing the active bleaching agent into a fluidized bed to form particles, spraying onto the particles an aqueous solution that comprises the water-soluble material, e.g. polyvinyl alcohol, to form the coating, and drying the coated particles. A description of this type of technology can be found in US2007093402.

The present inventors have observed that when the carbon atom that his bonded to the third carbon atom of the bleach catalyst having Formula (1) is not bonded to any hydrogen atom, then the bleach catalyst is stable against degradation. In other words, when the carbon atom that is bonded to the cyclic sulfone imine in the bleach catalyst having formula (1) is not bonded to any hydrogen atom, then the bleach catalyst is stable against degradation. The inventors have found that the presence of a proton (hydrogen atom) in the carbon atom of the R² group that is attached to the C═N bond leads to undesired yellowing of the catalyst, which is a clear sign of degradation of the catalyst.

Therefore, in an embodiment R² is an organic group in which the carbon atom that is bonded to the third carbon of the bleach catalyst is not bonded to any hydrogen atom.

In a preferred embodiment, R² is:
a branched alkyl or heteroalkyl having 4 to 16 carbon atoms, preferably 4 to 12 carbon atoms, more preferably 4 to 10 carbon atoms, even more preferably 4 to 8 carbon atoms;
a cycloalkyl-, cycloalkenyl-, cycloalkynyl-, or aryl-substituted linear or branched alkyl having 2 to 16 carbon atoms in the alkyl chain, preferably having 2 to 12 carbon atoms in the alkyl chain, more preferably having 2 to 10 carbon atoms in the alkyl chain, even more preferably 2 to 8 carbon atoms in the alkyl chain;
a branched or cyclic alkenyl having 3 to 16 carbon atoms, preferably having 3 to 12 carbon atoms, more preferably having 3 to 10 carbon atoms, even more preferably having 3 to 8 carbon atoms;
a heterocyclic alkenyl;
a cycloalkyl-, cycloalkenyl-, cycloalkynyl-, or aryl-substituted linear or branched alkenyl having 2 to 16 carbon atoms in the alkenyl chain, preferably having 2 to 12 carbon atoms in the alkenyl chain, more preferably having 2 to 10 carbon atoms in the alkenyl chain, even more preferably having 2 to 8 carbon atoms in the alkenyl chain;
a linear alkynyl having 2 to 16 carbon atoms, preferably having 2 to 12 carbon atoms, more preferably having 2 to 10 carbon atoms, even more preferably having 2 to 8 carbon atoms;
a branched alkynyl having 5 to 16 carbon atoms, preferably having 5 to 14 carbon atoms, more preferably having 5 to 12 carbon atoms, even more preferably having 5 to 10 carbon atoms;
a cycloalkyl-, cycloalkenyl-, cycloalkynyl-, or aryl-substituted linear or branched alkynyl having 2 to 16 carbon atoms in the alkynyl chain, preferably having 2 to 12 carbon atoms in the alkynyl chain, more preferably having 2 to 10 carbon atoms in the alkynyl chain, even more preferably having 2 to 8 carbon atoms in the alkynyl chain;
a substituted aryl,
an aryl with multiple or fused rings in which at least one ring is aromatic, or
a heteroaryl.

For example, when R² can be:
a branched alkyl or heteroalkyl having 4 to 16 carbon atoms, preferably 4 to 12 carbon atoms, more preferably 4 to 10 carbon atoms, even more preferably 4 to 8 carbon atoms, or
a cycloalkyl-, cycloalkenyl-, cycloalkynyl-, or aryl-substituted linear or branched alkyl having 2 to 16 carbon atoms in the alkyl chain, preferably having 2 to 12 carbon atoms in the alkyl chain, more preferably having 2 to 10 carbon atoms in the alkyl chain, even more preferably 2 to 8 carbon atoms in the alkyl chain;
then the carbon in R² that is bonded to the third carbon atom of the bleach catalyst having formula (1) is a quaternary carbon atom.

In a preferred embodiment, R² is:
a heterocyclic alkenyl,
a substituted aryl,
an aryl with multiple or fused rings in which at least one ring is aromatic, or
a heteroaryl;
preferably R² is a heterocyclic alkenyl or a heteroaryl;
more preferably R² is pyridyl, quinolyl, isoquinolinyl, acridinyl, pyrazinyl, quinoxalinyl, imidazolyl, benzimidazolyl, purinyl, pyrazolyl, indazolyl, pyrimidinyl, quinazolinyl, pyridazinyl, or cinnolinyl;
even more preferably R² is a pyridyl;
most preferably R² is 2-pyridyl or 3-pyridyl.

Preferred catalysts are those in which R¹ is Hydrogen and R² is 2-pyridyl or 3-pyridyl. In this case, the catalyst of formula 1 can be represented as follows:

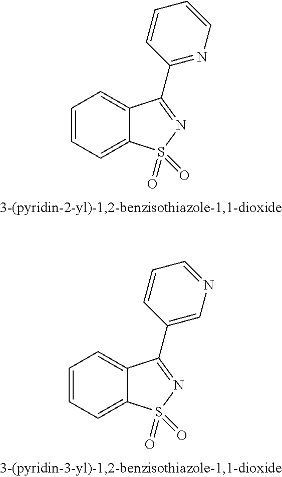 [SMILES: O=S1(=O)N=C(c2ccccn2)c2ccccc21 ; O=S1(=O)N=C(c2cccnc2)c2ccccc21]

### Preparation of Bleach Catalysts According to the Invention

The person skilled in the art will know how to prepare the bleach catalysts according to the invention, for which the reaction mechanism is:

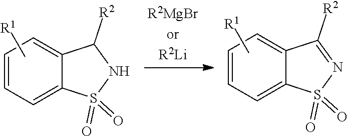

### Preparation of 3-(pyridin-2-yl)-1,2-benzisothiazole-1,1-dioxide

The bleach catalyst 3-(pyridin-2-yl)-1,2-benzisothiazole-1,1-dioxide can be prepared by reacting 1 eq. with a Grignard solution of the pyridine halide, added dropwise under cooling in a water-bath (ca.1.5 h). The suspension is stirred over night at room temperature. A sample quenched in saturated NH₄Cl shows nearly no starting material. The product is then obtained through recrystallisation from EtOH 1 eq. saccharin with 1 eq. Grignard solution of 2-pyridine halide and the solution is allowed to warm up at room temperature and stirred overnight. Thin layer chromatography analysis shows no starting material and NMR analysis confirms the purity of the sample.

The setup to obtain the desired molecule is as follow: 30 ml of Methyl tert-butyl ether (MTBE) and then a mixture of 15 ml 2N HCl and 20 ml saturated NaCl is added. The organic phase is separated and washed with 10 ml saturated NaCl. After drying with Na₂SO₄ the solvent is removed and to the residue (1.9 g) 10 ml EtOH are added and removed in vacuo. This is repeated once, and the off-white residue is crystallized from EtOH. The mother liquor contains the impurity contained in the starting material.

### Preparation of 3-(pyridine-3-yl)-1,2-benzisothiazole-1,1-dioxide

Similarly, 3-(pyridine-3-yl)-1,2-benzisothiazole-1,1-dioxide can be prepared by reacting 1 eq. with a Grignard solution of 3-pyridine halide, added dropwise under cooling in a water-bath (ca.1.5 h). The suspension is stirred over night at room temperature. A sample quenched in saturated NH₄Cl shows nearly no starting material. The product is then obtained through recrystallisation from EtOH eq. saccharin with 1 eq. Grignard solution of 3-pyridine halide. The setup is the same as described above.

### Preparation of 4-methoxy-3-methylbenzo[d]isothiazole 1,1-dioxide

For the preparation of a bleach catalyst in which R¹ is a methoxy group, such as in the case of 4-methoxy-3-methylbenzo[d]isothiazole 1,1-dioxide, the following procedure is provided. Starting from commercially available 3-Methoxy-benzenesulfonylchloride and tert-Butylamine, a tetrahydrofuran (THF) solution was made, and reactants were mixed at room temperature and stirred overnight. Hydrochloride is filtered off, washed and discarded with THF. The solvent is removed under reduced pressure giving pale yellow oil. Cyclohexane is added and crystallization starts after some time at room temperature.

Colorless crystals formed over-night, collected, washed with cyclohexane and dried under vacuum.

The obtained intermediate product is dissolved in THF, cooled down to −60° C. 2.2 eq. of n-butyllithium (2.5 M in hexane) were added dropwise and stream of carbon dioxide was passed into the flask and maintained for 1 h at room temperature after the reaction.

The workup protocol was as follows: the solvents were removed under reduced pressure and the rest was partitioned between 400 ml water and 200 ml MTBE. The phases were separated, and the aqueous phase was washed with MTBE. The aqueous phase was acidified using 2N HCl (to pH 1-2), and the precipitate was collected and washed with water and dried.

To obtain the methoxy-saccharine, the reaction mixture was heated at 60° C., the polyphosphoric acid was added, and the reaction mixture was stirred at 70° C. for 20 min, the reaction mixture turned deep yellow and stayed opaque. The workup was as follow: To the still warm suspension, ice was added and the precipitate that formed was collected and washed with water and dried in the form of a slightly greyish powder. The product was crystallization from EtOH to produce pure methoxy-saccharine.

This procedure can be modified for other R¹ benzenesulfonylchloride to deliver other R¹ modified-saccharine derivatives.

Once the methoxy-saccharine derivative is obtained, the procedure is similar to that described for other R² substituents: to obtain the 4-methoxy-3-methylbenzo[d]isothiazole 1,1-dioxide for example, 1 eq. of methyl Grignard was added, and the solution allowed to warm up at room temperature and stirred overnight. Thin layer chromatography analysis showed no starting material and NMR analysis confirmed the purity of the sample. The workup to obtain the desired molecule was as follow: MTBE and then a 3:4 mixture of 2N HCl and saturated NaCl was added. The organic phase was separated and washed with saturated NaCl. After drying with Na₂SO₄ the solvent was removed and EtOH was added to the residue and removed in vacuo. This was repeated once, and the off-white residue was crystallized from EtOH to obtain the pure 4-methoxy-3-methylbenzo[d]isothiazole 1,1-dioxide.

In a second aspect of the invention, it relates to a granule comprising the bleach catalyst according to the first aspect of the invention and a carrier.

The present inventors have observed that a granule comprising the bleach catalyst according to the first aspect of the invention and a carrier has a better catalytic activity than the bleach catalyst alone. Furthermore, the granule reduces or prevents deterioration of the bleach catalyst and allows for the incorporation of small quantities of the bleach catalyst into, e.g. cleaning compositions.

The granule can comprise between 0.1 wt. % and 40 wt. % of the bleach catalyst, based on the total weight of the granule, preferably between 0.2 wt. % and 10 wt. %, even more preferably between 0.5 wt. % and 5 wt. %.

The granule comprises a carrier, which can be selected from the group consisting of sulphates, carbonates, sesquicarbonates, silicates, aluminosilicates, zeolites, precipitated silicas, fumed silicas, and combinations thereof; preferably selected from the group consisting of sulphates, carbonates, sesquicarbonates, and combinations thereof; more preferably selected from the group consisting of the alkali metal salts of sulphates, carbonates, sesquicarbonates, and combinations thereof.

The granule may further comprise a binder. For example, the binder may include natural polymers such as starch, modified starch, carrageenan, gum arabic and guar gum and synthetic polymers such as polyethylene oxide, polyvinyl pyrrolidone, polyethylene glycol and polyethylene oxide/polypropylene oxide or others described in US2012028874. Also, anionically or nonionically modified celluloses with ether bonds can be employed as additional components of the coating material. Examples of cellulose ethers that can be used as component are carboxymethylcellulose (CMC), methyl cellulose (MC). ethyl cellulose (EC), hydroxyethyl cellulose (HEC) or hydroxypropyl cellulose (HPC). Other examples of cellulose ethers that can be used as component are mixed cellulose ethers. such as methyl ethyl cellulose (MEC), hydroxyethyl methyl cellulose (NEMC), hydroxypropyl methyl cellulose (HPMC), ethyl-hydroxyethyl cellulose or carboxymethyl hydroxyethyl cellulose.

The granule may comprise at least one surfactant; for example a non-ionic, anionic, cationic, amphoteric or zwitterionic surfactants or suitable mixtures thereof may be used. Suitable surfactants are described in Kirk Othmer's Encyclopaedia of Chemical Technology, 3rd Ed., Vol. 22, pp. 360-379, “Surfactants and Detersive Systems”.

In an embodiment the granule comprises at least one surfactant, preferably at least one non-ionic surfactant.

Examples of non-ionic surfactants are ethoxylated alcohols and ethoxylated alkyl phenols having the formula R¹(OCH₂CH₂)ₙOH; wherein R¹ is an aliphatic hydrocarbon radicals containing between 10 and 24 carbon atoms or alkyl phenyl radicals in which the alkyl groups contain from 10 to 22 carbon atoms; and n has an average value of from 1 to 10. Preferably, the one or more non-ionic surfactants have the formula R¹(OCH₂CH₂)ₙOH; wherein R¹ is an alkyl moiety having between 10 and 24 carbon atoms, preferably between 12 and 20 carbon atoms, more preferably between 12 and 18 carbon atoms; and n has an average value of from 1 to 10.

For example, when R¹ is defined as being a C₁₂₋₁₄ moiety, it is meant an alkyl moiety having between 12 and 14 carbon atoms. For example, when R¹ is defined as being a C₁₂₋₁₆ moiety, it is meant an alkyl moiety having between 12 and 16 carbon atoms. For example, when R¹ is defined as being a C₁₃ moiety, it is meant an alkyl moiety having 13 carbon atoms.

The at least one surfactant may comprise any conventional anionic surfactant or a mixture of them used in detergent products. These include, for example, the alkyl benzene sulfonic acids and their salts as well as alkoxylated or non-alkoxylated alkyl sulfate materials. The anionic surfactants may be present in acid form or in neutralized (e.g., salt) form. The anionic surfactants may be linear, branched, or a mixture thereof. An example of an anionic surfactant is sodium cumenesulphonate.

Examples of anionic surfactants are the alkali metal salts of C10-16 alkyl benzene sulphonic acids or C11-14 alkyl benzene sulphonic acids.

Another exemplary type of anionic surfactant is alkoxylated alkyl sulphate surfactants, such as ethoxylated alkyl sulphate surfactants. Such materials are also known as alkyl ether sulphates or alkyl polyethoxylate sulphates.

Alkyl ether sulphates are generally available in the form of mixtures comprising varying R′ chain lengths and varying degrees of ethoxylation. Frequently such mixtures also contain some non-ethoxylated alkyl sulphate (“AS”) materials. The granule may further comprise one or more additional ingredients such as polymers, plasticisers, anti-agglomeration agents, optical brightener, hueing agents, dye transfer inhibiting agents, bleaching agents, enzymes, and combinations thereof.

In a preferred embodiment, the granule has a particle size of between 200 μm and 2000 μm, preferably between 300 μm and 1500 μm, more preferably between 500 μm and 1200 μm.

In a third aspect, the invention relates to a method of preparing the granule according to the second aspect of the invention, wherein the method comprises the steps of:
a) Preparing a solution by dissolving the bleach catalyst in a solvent system,
b) Adding the solution prepared in step a) to the carrier,
c) Allowing the solvent system to evaporate partially or completely, wherein the solvent system has a dielectric constant below 80, a vapour pressure above 5 kPa and a boiling point below 100° C.

The solvent system may comprise one or more substances with a low molecular weight, for example, one or more substances with a molecular weight below 180 Dalton. In an embodiment, the solvent system comprises one or more solvents selected from the group consisting of water, methanol, ethanol, propylene carbonate, acetone, acetonylacetone, diacetone alcohol, ethyl acetate, 2-propanol, ethylene glycol, propylene glycol, glycerin, diethylene glycol, dipropylene glycol, monomethyl ether, dimethylformamide, and combinations thereof.

In a preferred embodiment, the solvent system comprises less than 80% water, based on the total weight of the solvent system, preferably less than 50%, even more preferably less than 20%.

The solvent system can have:
a dielectric constant below 40 and/or
a vapour pressure above 7 kPa, preferably above 10 kPa, and/or
a boiling point below 80° C., preferably below 70° C.

The solvent system can further comprise a surfactant, preferably a non-ionic surfactant. It will be understood that the surfactants described in the second aspect of the invention are applicable in the method according to the third aspect of the invention. The present inventors have observed that when a surfactant is added to the solvent system, the amount of solvent system needed to dissolve the bleach catalyst in the solvent system is reduced; in addition, the catalytic activity of the bleach catalyst is increased.

The person skilled in the art will understand that it is possible to prepare the granule according to the second aspect of the invention following an alternative method, for example:
i. Grinding the bleach catalyst,
ii. Grinding the carrier,
iii. Adding a binder to the grinded bleach catalyst and carrier to obtain an agglomerate or granules.
iv. Optionally grinding the agglomerate to obtain granules.

In a fourth aspect, the invention relates to a cleaning composition comprising the bleach catalyst according to the first aspect of the invention and/or the granule according to the second aspect of the invention, at least one bleaching agent and at least one bleach activator.

In an embodiment, the at least one bleaching agent is partially or totally contained in the granule.

In another embodiment, the at least one bleach activator is partially or totally contained in the granule.

In a further embodiment, the at least one bleaching agent and the at least one bleach activator are partially or totally contained in the granule.

The composition can be a hard-surface cleaning composition or a soft-surface cleaning composition, preferably an automatic dishwashing composition or a laundry composition, more preferably a laundry detergent composition or a laundry additive composition, most preferably a laundry additive composition.

In an embodiment, the composition comprises between 0.001 wt. % and 10 wt. % of the bleach catalyst, based on the total weight of the composition, preferably between 0.005 wt. % and 5 wt. %, more preferably between 0.01 wt. % and 1 wt. %, most preferably between 0.025 wt. % and 0.5 wt. %. The bleach catalyst can be partially or totally contained in the granule according to the second aspect of the invention. The at least one bleaching agent may comprise at least one source of active oxygen selected from the group consisting of an inorganic peroxide, an organic peracid, hydrogen peroxide, and combinations thereof; preferably selected from the group consisting of sodium percarbonate, sodium perborate, ε-phthalimidoperoxy-hexanoic acid (PAP), peracetic acid, potassium peroxymonosulfate (KMPS), and combinations thereof; most preferably wherein said at least one bleaching agent is sodium percarbonate.

The composition may then comprise between 1 wt. % and 80 wt. % of the at least one bleaching agent, based on the total weight of the composition, preferably between 5 wt. % and 70 wt. %, more preferably between 10 wt. % and 65 wt. %, even more preferably between 15 wt. % and 60 wt. %. For example, when the cleaning composition is a laundry additive composition, it may comprise between 18 wt. % and 58 wt. % of the at least one bleaching agent. The at least one source of active oxygen can be partially or totally contained in the granule according to the second aspect of the invention.

The at least one bleach activator may be selected from the group consisting of tetraacetylethylenediamine (TAED), acetylated triazine derivatives, acetylated glycoluriles, acylimides, acetylated glucose, acetylated glycerol, acetylated phenolsulfonates, acetylated phenol carbonic acids, sodium nonanoyloxybenzenesulfonate (NOBS), 4-decanoyloxybenzoic Acid (DOBA), carbonic acid anhydrides, acetylated sugar derivatives, N-acetylated lactams, and combinations thereof; preferably said at least one bleach activator is tetra acetylethylenediamine (TAED).

The composition may then comprise between 0.1 wt. % and 20 wt. % of the at least one bleach activator, based on the total weight of the composition, preferably between 0.25 wt. % and 15 wt. %, more preferably between 0.5 wt. % and 10 wt. %, most preferably between 1 wt. % and 5 wt. %. The at least one bleach activator can be partially or totally contained in the granule according to the second aspect of the invention.

The composition according to the invention allows for the incorporation of enzymes. For example, the cleaning composition can comprise at least one enzyme selected from the group consisting of a mannanase, a lipase, an amylase, a cellulase, a protease, and combinations thereof. For example, the cleaning composition can comprise between 0.001 wt. % and 10 wt. % of the at least one enzyme, based on the total weight of the composition, preferably between 0.001 wt. % and 8 wt. %, more preferably between 0.001 and 6 wt. %, even more preferably between 0.001 and 4 wt. %, most preferably between 0.001 wt. % and 2 wt. %. The at least one enzyme can be partially or totally contained in the granule according to the second aspect of the invention.

The compositions of the present invention may take any form, e.g., solid, liquid, gel, powder or mixtures thereof. Preferably, the compositions will be in the form of a solid or a powder.

Preferably the composition further comprises a rheology modifier agent, a fragrance, a phase stabilizer, a dye, a softening agent, a chelating agent, an anti-bacterial agent, a transition metal compound, an anti-foaming agent, a preservative, one or more surfactants, one or more fillers, a dye-transfer inhibitor, an optical brightener, or combinations thereof.

The composition may comprise one or more non-ionic surfactants. The non-ionic surfactants described in the second aspect of the invention can be used in the composition according to the fourth aspect of the invention. The amount of said one or more non-ionic surfactants in the composition may be between 0.01 wt. % and 10 wt. %, based on the total weight of the composition, preferably between 0.05 wt. % and 5 wt. %, more preferably between 0.1 wt. % and 1.0 wt. %. The one or more non-ionic surfactants can be partially or totally contained in the granule according to the second aspect of the invention.

In a preferred embodiment, the composition comprises between 0.01 wt. % and 10 wt. % of one or more non-ionic surfactants, based on the total weight of the composition, preferably between 0.05 wt. % and 5 wt. %, more preferably between 0.1 wt. % and 1.0 wt. %; wherein said one or more non-ionic surfactants have the formula R¹(OCH₂CH₂)ₙOH; wherein R¹ is an alkyl moiety having between 12 and 16 carbon atoms, and n has an average value of from 3 to 8, preferably between 4 and 6. The one or more non-ionic surfactants can be partially or totally contained in the granule according to the second aspect of the invention.

The composition may optionally contain a filler. Suitable fillers include bicarbonates and carbonates of metals, such as alkali metals and alkaline earth metals. Examples include sodium carbonate, sodium bicarbonate, calcium carbonate, calcium bicarbonate, magnesium carbonate, magnesium bicarbonate and sesqui-carbonates of sodium, calcium and/or magnesium. Other examples include metal carboxy glycine and metal glycine carbonate. Chlorides, such as sodium chloride; citrates; and sulphates, such as sodium sulphate, calcium sulphate and magnesium sulphate, may also be employed. Preferably the composition comprises at least one filler selected from the group comprising alkyl carbonate, alkyl sulphate and combinations thereof; more preferably selected from the group comprising sodium carbonate, sodium sulphate and combinations thereof.

It will be understood that a filler and a carrier in the present invention are two different ingredients. When a filler is present, the composition may comprise between 0.1 wt. % and 80 wt. % of said at least one filler, based on the total weight of the composition, preferably between 10 wt. % and 75 wt. %, more preferably between 20 wt. % and 70 wt. %.

The composition may comprise an optical brightener, preferably selected from the group comprising stilbene, benzidine, benzothiazole, benzimidazole, benzoxazole, coumarin, pyrazoline, naphtalimide, naphtoxazole, distyryl-biphenyl, benzonitrile, benzopyrazole, including their derivatives and substituted compounds, and combinations thereof. Suitable optical brighteners include 4,4′-bis(2-sulphostyryl)diphenyl, 7-Diethylamino-4-methylcoumarin, benzoxazole, 2,2′-(1,2-ethenediyl) bis[5-methyl]including their substituted compounds, 4,4′-bis (triazine-2ylamino) stilbene-2,2′-disulphonic acid, mono (azol-2-yl) stilbene and bis(azol-2yl) stilbene; styryl derivatives of benzene and biphenyl, such as 1,4-bis (styryl) benzene, 4,4′-bis (styryl) benzene, 4,4′-bis-(styryl) biphenyl, 4,4′-bis (sulphostryryl) biphenyl sodium salt; pyrazolines such as 1, 3-diphenyl-2-pyrazoline; bis(benzene-2-yl) derivatives, bis(benzoxazol-2-yl) derivatives and bis(benzimidazol-2-yl) derivatives; 2-(benzofuran-2-yl) benzimidazole; coumarins such as 4-methyl-7-hydroxy-coumarin or 4-methyl-7-diethylaminocoumarin; carbostyrils; naphthalimides; dibenzothiophene5,5-dioxide; pyrene; or pyridotriazole derivatives and combinations thereof. The optical brightener can be partially or totally contained in the granule according to the second aspect of the invention.

When an optical brightener is present, the composition may comprise between 0.001 wt. % and 1 wt. % of at least one optical brightener, based on the total weight of the composition, preferably between 0.01 wt. % and 0.5 wt. %, more preferably between 0.05 wt. % and 0.25 wt. %. The optical brightener can be partially or totally contained in the granule according to the second aspect of the invention.

The composition may further comprise a dye-transfer inhibitor. Preferred dye-transfer inhibitors (sometimes called dye anti-redisposition agents or soil suspending agents) include polyvinylalcohol, fatty amides, sodium carboxylmethyl cellulose, hydroxypropyl methyl cellulose, polyvinylpyrollidone, polyvinylimidazole, polyvinyloxazolidone, polyamine N-oxide polymers and copolymers or N-vinylpyrollidone and N-vinylimidazole. The dye-transfer inhibitor can be partially or totally contained in the granule according to the second aspect of the invention.

When a dye-transfer inhibitor is present, the composition may comprise between 0.001 wt. % and 1.5 wt. % of at least one dye transfer inhibitor, based on the total weight of the composition, preferably between 0.01 wt. % and 1.0 wt. %, more preferably between 0.05 wt. % and 0.75 wt. %. The dye-transfer inhibitor can be partially or totally contained in the granule according to the second aspect of the invention.

The composition may comprise one or more polymers having a molecular weight between 1000 to 200000, preferably between 1000 to 150000, more preferably between 1500 to 50000, even more preferably between 1500 to 10000. In an embodiment the composition comprises carboxylates with the above-mentioned molecular weights, in particular carboxylates/carboxylic acids such as polyacrylates/polyacrylic acid and derivatives/copolymers thereof. Suitable carboxylates include monomeric carboxylates/carboxylic acids such as citric acid/citrate, maleic acid/maleate and derivatives thereof. Suitable polymeric polyols for coating include hydrolysed polyacetates (wherein a portion of the acetate moieties are hydrolysed to hydroxyl moieties, e.g. to polyvinyl alcohol) and derivatives thereof. In an aspect, the polymer for the polymeric matrix is polyvinylpyrrolidone (PVP).

Non-limiting examples of commercially available polyacrylates are Acusol® 448 (acrylic acid/maleic acid copolymer) and Acusol® 445 (acrylic acid polymer, 45 percent active), available from Dow Chemical.

The one or more polymers can be partially or totally contained in the granule according to the second aspect of the invention. For example, the one or more polymers can be used to coat the granule according to the second aspect of the invention.

The composition may comprise one or more plasticisers. For example, polyols such as sugars, sugar alcohols, or polyethylene glycols (PEGs), urea, glycol, propylene glycol or other known plasticizers such as triethyl citrate, dibutyl or dimethyl phthalate, polyethylene glycerin, sorbitol, tribuyl citrate, dibutyl sebecate and/or polysorbates. The one or more plasticisers can be partially or totally contained in the granule according to the second aspect of the invention.

In a fifth aspect, the invention relates to the use of the cleaning composition of the fourth aspect of the invention for the removal of stains, preferably stains comprising a polyphenolic compound, more preferably wherein the stains comprise turmeric and/or curry stains and/or for the removal of malodour from a garment.

The composition can be used for the removal of stains from a garment, wherein the garment is made of cotton, synthetic materials, or a combination thereof, preferably wherein said synthetic material is polyester.

In a sixth aspect, the invention relates to a method of washing clothes (or remove stains), wherein the method comprises providing the composition according to the fourth aspect of the invention to a laundry washing machine and performing the washing at a temperature lower than 60° C. and for a time shorter than 120 minutes.

In one embodiment, the washing in the method is performed a temperature lower than 40° C., preferably lower than 30° C., more preferably lower than 25° C.

In another embodiment, the washing in the method is carried out for a time shorter than 60 minutes, preferably shorter than 45 minutes, more preferably shorter than 40 minutes, even more preferably shorter than 30 minutes.

When carrying out the method according to the sixth aspect of the invention preferably between 0.5 g and 20 g of the composition are added to the laundry washing machine per litre of washing liquor, more preferably between 1 g and 15 g, even more preferably between 2 g and 10 g, most preferably between 2.5 g and 7.5 g.

It would be understood that when the clothes are washed in the washing machine, preferably stains and malodour are removed.

The foregoing aspects may be freely combined with any of the foregoing aspects disclosed herein.

The invention will be further described, by way of example, with the reference to the following non-limiting embodiments.

### CLAUSES

1. a bleach catalyst having formula (1):

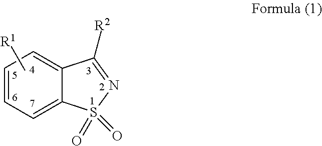

wherein:
   R¹ is H, halogen, hydroxy, amino, nitro, cyano, carboxy, alkyl, C₁-C₁₀-alkoxy, C₁-C₁₀-alkylamino, phenyl, substituted phenyl, or a heterocyclic ring;
   R² is an organic group comprising a carbon atom that is bonded to the third carbon of the bleach catalyst,
   the partial charge density of the C═N bond of the bleach catalyst is less than −0.180, and
   the ClogP of the bleach catalyst is lower than 2.
2. The bleach catalyst according to clause 1, wherein the ClogP of the bleach catalyst is lower than 1.8, preferably lower than 1.5, more preferably lower than 1, even more preferably between 0.2 and 0.90, most preferably between 0.3 and 0.85.
3. The bleach catalyst according to clause 1 or clause 2, wherein the partial charge density of the C═N bond is less than −0.190, preferably less than −0.195, more preferably less than −0.200.
4. The bleach catalyst according to any of the preceding clauses, wherein R¹ is H, hydroxy, or C₁-C₁₀-alkoxy, preferably H.
5. The bleach catalyst according to any of the preceding clauses, wherein R² is alkyl, heteroalkyl, alkenyl, alkynyl, aryl, substituted aryl, aryl with multiple or fused rings in which at least one ring is aromatic, heteroaryl, or heterocyclic ring.
6. The bleach catalyst according to any of the preceding clauses, wherein R² is an organic group in which the carbon atom that is bonded to the third carbon of the bleach catalyst is not bonded to any hydrogen atom.
7. The bleach catalyst according to any of the preceding clauses, wherein R² is:
   a branched alkyl or heteroalkyl having 4 to 16 carbon atoms, preferably 4 to 12 carbon atoms, more preferably 4 to 10 carbon atoms, even more preferably 4 to 8 carbon atoms;
   a cycloalkyl-, cycloalkenyl-, cycloalkynyl-, or aryl-substituted linear or branched alkyl having 2 to 16 carbon atoms in the alkyl chain, preferably having 2 to 12 carbon atoms in the alkyl chain, more preferably having 2 to 10 carbon atoms in the alkyl chain, even more preferably 2 to 8 carbon atoms in the alkyl chain;
   a branched or cyclic alkenyl having 3 to 16 carbon atoms, preferably having 3 to 12 carbon atoms, more preferably having 3 to 10 carbon atoms, even more preferably having 3 to 8 carbon atoms;
   a heterocyclic alkenyl;
   a cycloalkyl-, cycloalkenyl-, cycloalkynyl-, or aryl-substituted linear or branched alkenyl having 2 to 16 carbon atoms in the alkenyl chain, preferably having 2 to 12 carbon atoms in the alkenyl chain, more preferably having 2 to 10 carbon atoms in the alkenyl chain, even more preferably having 2 to 8 carbon atoms in the alkenyl chain;
   a linear alkynyl having 2 to 16 carbon atoms, preferably having 2 to 12 carbon atoms, more preferably having 2 to 10 carbon atoms, even more preferably having 2 to 8 carbon atoms;
   a branched alkynyl having 5 to 16 carbon atoms, preferably having 5 to 14 carbon atoms, more preferably having 5 to 12 carbon atoms, even more preferably having 5 to 10 carbon atoms;
   a cycloalkyl-, cycloalkenyl-, cycloalkynyl-, or aryl-substituted linear or branched alkynyl having 2 to 16 carbon atoms in the alkynyl chain, preferably having 2 to 12 carbon atoms in the alkynyl chain, more preferably having 2 to 10 carbon atoms in the alkynyl chain, even more preferably having 2 to 8 carbon atoms in the alkynyl chain;
   a substituted aryl,
   an aryl with multiple or fused rings in which at least one ring is aromatic, or
   a heteroaryl.
8. The bleach catalyst according to clause 7, wherein R² can be:
   a branched alkyl or heteroalkyl having 4 to 16 carbon atoms, preferably 4 to 12 carbon atoms, more preferably 4 to 10 carbon atoms, even more preferably 4 to 8 carbon atoms, or
   a cycloalkyl-, cycloalkenyl-, cycloalkynyl-, or aryl-substituted linear or branched alkyl having 2 to 16 carbon atoms in the alkyl chain, preferably having 2 to 12 carbon atoms in the alkyl chain, more preferably having 2 to 10 carbon atoms in the alkyl chain, even more preferably 2 to 8 carbon atoms in the alkyl chain;
   then the carbon in R² that is bonded to the third carbon atom of the bleach catalyst having formula (1) is a quaternary carbon atom.
9. The bleach catalyst according to clause 7, wherein R² is:
   a heterocyclic alkenyl,
   a substituted aryl,
   an aryl with multiple or fused rings in which at least one ring is aromatic, or
   a heteroaryl.
10. The bleach catalyst according to clause 9, wherein R² is a heterocyclic alkenyl or a heteroaryl.
11. The bleach catalyst according to clause 10, wherein R² is pyridyl, quinolyl, isoquinolinyl, acridinyl, pyrazinyl, quinoxalinyl, imidazolyl, benzimidazolyl, purinyl, pyrazolyl, indazolyl, pyrimidinyl, quinazolinyl, pyridazinyl, or cinnolinyl.
12. The bleach catalyst according to clause 11, wherein R² is a pyridyl, preferably 2-pyridyl or 3-pyridyl.
13. The bleach catalyst according to clause 12, wherein R¹ is Hydrogen and R² is 2-pyridyl or 3-pyridyl.
14. A granule comprising the bleach catalyst according to any of the preceding clauses and a carrier.
15. The granule according to clause 14, wherein the carrier is selected from the group consisting of sulphates, carbonates, sesquicarbonates, silicates, aluminosilicates, zeolites, precipitated silicas, fumed silicas, and combinations thereof; preferably selected from the group consisting of sulphates, carbonates, sesquicarbonates, and combinations thereof; more preferably selected from the group consisting of the alkali metal salts of sulphates, carbonates, sesquicarbonates, and combinations thereof.
16. The granule according to clause 14 or clause 15, wherein the granule comprises between 0.1 wt. % and 40 wt. % of the bleach catalyst, based on the total weight of the granule, preferably between 0.2 wt. % and 10 wt. %, even more preferably between 0.5 wt. % and 5 wt. %.
17. The granule according to any of the clauses 14-16, wherein the granule further comprises at least one surfactant.
18. The granule according to clause 17, wherein the at least one surfactant is selected from the group consisting of non-ionic, anionic, cationic, amphoteric, zwitterionic surfactants, and combinations thereof, preferably wherein the at least one surfactant is a non-ionic surfactant.
19. The granule according to any of the clauses 14-18, wherein the granule further comprises a binder.
20. The granule according to clause 19, wherein the binder is selected from the group consisting of natural polymers, synthetic polymers, and combinations thereof.
21. The granule according to any of the clauses 14-20, wherein the granule further comprises at least one bleaching agent preferably comprising at least one source of active oxygen.
22. The granule according to claim 21, wherein the at least one source of active oxygen is selected from the group consisting of an inorganic peroxide, an organic peracid, hydrogen peroxide, and combinations thereof; preferably selected from the group consisting of sodium percarbonate, sodium perborate, ε-phthalimidoperoxy-hexanoic acid (PAP), peracetic acid, potassium peroxymonosulfate (KMPS), and combinations thereof; most preferably wherein said at least one bleaching agent is sodium percarbonate.
23. The granule according to any of the clauses 14-22, wherein the granule further comprises at least one bleach activator, preferably selected from the group consisting of tetraacetylethylenediamine (TAED), acetylated triazine derivatives, acetylated glycoluriles, acylimides, acetylated glucose, acetylated glycerol, acetylated phenolsulfonates, acetylated phenol carbonic acids, sodium nonanoyloxybenzenesulfonate (NOBS), 4-decanoyloxybenzoic Acid (DOBA), carbonic acid anhydrides, acetylated sugar derivatives, N-acetylated lactams, and combinations thereof; preferably said at least one bleach activator is tetra acetylethylenediamine (TAED).
24. The granule according to any of the clauses 14-23, wherein the granule further comprises at least one enzyme selected from the group consisting of a mannanase, a lipase, an amylase, a cellulase, a protease, and combinations thereof.
25. The granule according to any of the clauses 14-24, wherein the granule further comprises at least one optical brightener.
26. The granule according to any of the clauses 14-25, wherein the granule further comprises at least one dye-transfer inhibitor.
27. The granule according to any of the clauses 14-26, wherein the granule further comprises one or more polymers having a molecular weight between 1000 to 200000, preferably between 1000 to 150000, more preferably between 1500 to 50000, even more preferably between 1500 to 10000; wherein the polymers comprise carboxylates, preferably polyacrylates.
28. The granule according to any of the clauses 14-27, wherein the granule further comprises one or more plasticisers.
29. The granule according to any of the clauses 14-28, wherein the granule further comprises a coating which is the most external layer of the granule.
30. The granule according to any of the clauses 14-29, wherein the granule has a particle size of between 200 μm and 2000 μm, preferably between 300 μm and 1500 μm, more preferably between 500 μm and 1200 μm.
31. A method of preparing the granule according to any of the clauses 14-30, wherein the method comprises the steps of:
   a) Preparing a solution by dissolving the bleach catalyst in a solvent system,
   b) Adding the solution prepared in step a) to the carrier,
   c) Allowing the solvent system to evaporate partially or completely,
wherein the solvent system has a dielectric constant below 80, a vapour pressure above 5 kPa and a boiling point below 100° C.
32. The method according to clause 31, wherein the solvent system comprises one or more solvents selected from the group consisting of water, methanol, ethanol, propylene carbonate, acetone, acetonylacetone, diacetone alcohol, ethyl acetate, 2-propanol, ethylene glycol, propylene glycol, glycerine, diethylene glycol, dipropylene glycol monomethyl ether and dimethylformamide.
33. The method according to clause 32, wherein the solvent system further comprises at least one surfactant.
34. The method according to clause 33, wherein the at least one surfactant is selected from the group consisting of non-ionic, anionic, cationic, amphoteric, zwitterionic surfactants, and combinations thereof, preferably wherein the at least one surfactant is a non-ionic surfactant.
35. The method according to any of the clauses 31-34, wherein the solvent system has a dielectric constant of below 40.
36. The method according to any of the clauses 31-35, wherein the solvent system has a vapour pressure above 7 kPa, preferably above 10 kPa.
37. The method according to any of the clauses 31-36, wherein the solvent system has a boiling point below 80° C., preferably below 70° C.
38. A method of preparing the granule according to any of the clauses 14-30, wherein the method comprises the steps of:
   i. Grinding the bleach catalyst,
   ii. Grinding the carrier,
   iii. Adding a binder to the grinded bleach catalyst and carrier to obtain an agglomerate or granules.
   iv. Optionally grinding the agglomerate to obtain granules.
39. A cleaning composition comprising the bleach catalyst according to any of the clauses 1-13, at least one bleaching agent and at least one bleach activator.
40. A cleaning composition comprising the granule according to any of the clauses 14-30.
41. The cleaning composition according to clause 40, wherein the composition comprises at least one bleaching agent and at least one bleach activator; wherein the at least one bleaching agent and/or the at least one bleach activator is/are partially or totally contained in the granule.
42. A cleaning composition comprising the bleach catalyst according to any of the clauses 1-13, the granule according to any of the clauses 14-30, at least one bleaching agent and at least one bleach activator, wherein the at least one bleaching agent and/or the at least one bleach activator is/are partially or totally contained in the granule.
43. The cleaning composition according to any of the clauses 39-42, wherein the composition is a hard-surface cleaning composition or a soft-surface cleaning composition, preferably an automatic dishwashing composition or a laundry composition, more preferably a laundry detergent composition or a laundry additive composition, most preferably a laundry additive composition.
44. The cleaning composition according to any of the clauses 39-43, wherein the composition comprises between 0.001 wt. % and 10 wt. % of the bleach catalyst, based on the total weight of the composition, preferably between 0.005 wt. % and 5 wt. %, more preferably between 0.01 wt. % and 1 wt. %, most preferably between 0.025 wt. % and 0.5 wt. %, preferably wherein the bleach catalyst is partially or totally contained in the granule.
45. The cleaning composition according to any of the clauses 39-44, wherein the at least one bleaching agent comprises at least one source of active oxygen selected from the group consisting of an inorganic peroxide, an organic peracid, hydrogen peroxide, and combinations thereof; preferably selected from the group consisting of sodium percarbonate, sodium perborate, ε-phthalimidoperoxy-hexanoic acid (PAP), peracetic acid, potassium peroxymonosulfate (KMPS), and combinations thereof; most preferably wherein said at least one bleaching agent is sodium percarbonate, preferably wherein the at least one bleaching agent is partially or totally contained in the granule.
46. The cleaning composition according to any of the clauses 39-45, wherein the at least one bleach activator is selected from the group consisting of tetraacetylethylenediamine (TAED), acetylated triazine derivatives, acetylated glycoluriles, acylimides, acetylated glucose, acetylated glycerol, acetylated phenolsulfonates, acetylated phenol carbonic acids, sodium nonanoyloxybenzenesulfonate (NOBS), 4-decanoyloxybenzoic Acid (DOBA), carbonic acid anhydrides, acetylated sugar derivatives, N-acetylated lactams, and combinations thereof; preferably said at least one bleach activator is tetra acetylethylenediamine (TAED), preferably wherein the at least one bleach activator is partially or totally contained in the granule.
47. Use of the cleaning composition according to any of the clauses 39-46 for the removal of stains, preferably stains comprising a polyphenolic compound, more preferably wherein the stains comprise turmeric and/or curry stains and/or for the removal of malodour from a garment.
48. Use according to clause 47 for the removal of stains from a garment, wherein the garment is made of cotton, synthetic materials or a combination thereof, preferably wherein said synthetic material is polyester.
49. A method of washing clothes, wherein the method comprises providing the composition according to any of the clauses 39-46 to a laundry washing machine and performing the washing:
   at a temperature lower than 60° C., preferably lower than 40° C., more preferably lower than 30° C., even more preferably lower than 25° C.; and
   for a time of less than 120 minutes, preferably less than 60 minutes, more preferably less than 45 minutes, even more preferably less than 40 minutes, most preferably less than 30 minutes.

### EXAMPLES

### Partition Coefficient ClogP and Partial Charge Density of the C═N Bond

The partition coefficient (ClogP) and the partial charge density of the C═N bond of bleach catalysts having the structures shown in Table 2 was determined using the methods described in this disclosure. The person skilled in the art will know how to synthesise the catalysts shown below starting from saccharine.

TABLE 2
Bleach catalysts

| Bleach catalyst | Name | Structure |
|---|---|---|
| A | 3-methyl-1,2-benzisothiazole-1,1-dioxide | 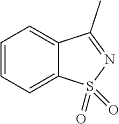 |
| B | 3-phenyl-1,2-benzisothiazole-1,1-dioxide | 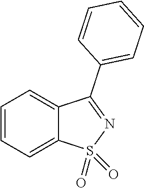 [SMILES: O=S1(=O)N=C(c2ccccc2)c2ccccc21] |
| C | 3-tert-butyl-1,2-benzisothiazole-1,1- dioxide | 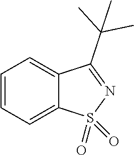 |
| D | 5-hydroxy-3-ethylbenzo[d]isothiazole 1,1- dioxide | 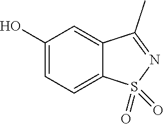 |
| E | 5-methoxy-3- methylbenzo[d]isothiazole1, 1-dioxide | 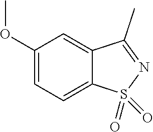 |
| 1 | 3-(33yridine-2-yl)-1,2-benzisothiazole- 1,1-dioxide | 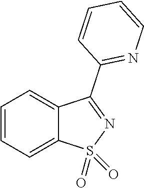 [SMILES: O=S1(=O)N=C(c2ccccn2)c2ccccc21] |
| 2 | 3-(pyridine-3-yl)-1,2-benzisothiazole-1,1- dioxide | 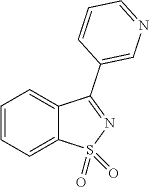 [SMILES: O=S1(=O)N=C(c2cccnc2)c2ccccc21] |
| 3 | 4-hydroxy-3-methylbenzo[d]isothiazole 1,1-dioxide | 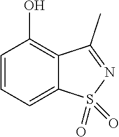 |
| 4 | 4-methoxy-3-methylbenzo[d]isothiazole 1,1-dioxide | 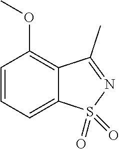 |

As previously mentioned, the present inventors have surprisingly observed that the performance of a bleach catalyst is directly linked with its octanol/water partition coefficient (ClogP) and the partial charge density of the C═N bond.

These parameters have been determined according to the methods shown above, and the ClogP values and the partial charges of the C═N bond (imine bond) of several bleach catalysts are shown in Table 3.

TABLE 3
Octanol/Water partition coefficient (ClogP) and partial
charge of the C═N bond (imine bond) of bleach catalysts

| Bleach | | Partial Charge | Partial Charge | Partial Charge |
|---|---|---|---|---|
| catalyst | ClogP | C | N | C═N |
| A | 0.82 | −0.483 | 0.306 | −0.177 |
| B | 2.25 | −0.465 | 0.257 | −0.208 |
| C | 2.06 | −0.485 | 0.286 | −0.199 |
| D | 0.16 | −0.4822 | 0.3057 | −0.1765 |
| E | 0.74 | −0.4836 | 0.3061 | −0.1775 |
| 1 | 0.76 | −0.458 | 0.255 | −0.203 |
| 2 | 0.76 | −0.465 | 0.258 | −0.207 |
| 3 | 0.16 | −0.5156 | 0.3060 | −0.2096 |
| 4 | 0.74 | −0.5141 | 0.3070 | −0.2071 |

### Stability of Bleach Catalysts

The stability of bleach catalysts having the structures shown in Table 2 was determined by preparing solutions of these compounds and measuring their UV-vis absorbance.

### Samples Preparation

A solution of each compound was prepared by dissolving each compound in a mixture of 50 wt. % water and 50 wt. % acetone. The concentration of each solution was 0.28 mM.

The pH of each solution was set to 11 by adding a diluted solution of sodium hydroxide.

### UV-Vis Absorbance Measurement

The UV-vis absorption of each solution was measured just after preparing the samples (t=0), then at 2 h and 48 h using a UV-Vis Evolution 220 FC-1135 spectrophotometer. Table 4 shows the absorption values of each sample at a wavelength of 445 nm and the appearance of the samples under alkaline conditions.

TABLE 4
Absorbance of solutions of bleach catalysts at 445 nm

| | Bleach | Absorption at 445 nm | | Appearance | |
|---|---|---|---|---|---|
| Solution | catalyst | t = 0 | t = 48 h | t = 0 | t = 48 h |
| Solution A | A | <0.05 | 0.37 | Transparent | Yellowing |
| Solution B | B | <0.05 | <0.05 | Transparent | Transparent |
| Solution C | C | <0.05 | <0.05 | Transparent | Transparent |
| Solution 1 | 1 | <0.05 | <0.05 | Transparent | Transparent |
| Solution 2 | 2 | <0.05 | <0.05 | Transparent | Transparent |

Table 4 clearly shows that the solution containing a bleach catalyst in which the carbon atom that is bonded to the carbon atom in position 3 of the bleach catalyst, i.e. bonded to the cyclic sulfone imine, is bonded to a hydrogen atom (bleach catalyst A) gets coloured/yellow after some time, for example, after 48 h. This is further evidenced by the UV-Vis absorption measurements, in which the solution absorbs more light at 445 nm after 48 h. This instability of the catalyst is a relevant issue, as performance and sensorial aspects of the product are affected.

### Preparation of Granule

Granule 1 without Surfactant

### Materials:

Acetone,
Bleach catalyst 1
Granular sodium sulphate with an average particle size between 500 μm and 750 μm, where at least 85% of the particles have a particle size above 250 μm and only 30% of the particles have a particle size above 750 μm.

50 mL of acetone and 2 g of bleach catalyst 1 were added to a closed container and agitated using a magnetic stirrer until the bleach catalyst was fully dissolved. The solution containing the bleach catalyst was poured onto 98 g of granular sodium sulphate. The mixture of sodium sulphate and the dissolved catalyst were agitated until all solvent evaporated.

The obtained granules were kept at 20-25° C., i.e. room temperature, for 24 hours.

Granule 1 with Surfactant

### Materials:

Acetone,
Bleach catalyst 1
C12-16 non-ionic surfactant with 7 moles of ethylene oxide, commercially available as ROKAnol L7A
Granular sodium sulphate with an average particle size between 500 μm and 750 μm, where at least 85% of the particles have a particle size above 250 μm and only 30% of the particles have a particle size above 750 μm.

25 mL of acetone and 0.06 g of the non-ionic surfactant were added to a closed container and agitated using a magnetic stirrer until a clear solution was formed, i.e. the solvent system. 2 g of bleach catalyst 1 were added to a closed container with the solvent system and agitated using a magnetic stirrer until the bleach catalyst was fully dissolved. The solution containing the bleach catalyst was poured onto 98 g of granular sodium sulphate. The mixture of sodium sulphate and the dissolved catalyst were agitated until all solvent evaporated.

The obtained granules were kept at 20-25° C., i.e. room temperature, for 24 hours.

### Effect of Surfactant on the Solubility of the Bleach Catalyst

### Solvents:

Acetone,
Ethyl acetate,
Tetrahydrofuran.

### Surfactant:

C12-16 non-ionic surfactant with 7 moles of ethylene oxide, commercially available as ROKAnol L7A.

The amount of solvent needed to dilute 0.2 g of bleach catalyst 1 was determined. Subsequently solvent systems containing a solvent and 0.06 wt. % of surfactant, based on the total weight of the solvent system, were prepared, and the amount of solvent system needed to dilute 0.2 g of bleach catalyst 1 was determined. It will be recognised that for the preparation of a supported catalyst on a carrier it is necessary to dilute the bleach catalyst in a solvent system.

The results of the amount of solvent system without surfactant and solvent system with surfactant needed to dissolve 0.2 g of bleach catalyst 1 are shown in Table 5.

TABLE 5
Amount of solvent system without surfactant and
solvent system with 0.06 wt. % of surfactant
needed to dissolve 0.2 g of bleach catalyst 1.

| Solvent system | Amount (g) |
|---|---|
| Acetone | 5 |
| Acetone with 0.06 wt. % surfactant | 2.5 |
| Ethyl acetate | 13 |
| Ethyl acetate with 0.06 wt. % surfactant | 11 |
| Tetrahydrofuran | 3.5 |
| Tetrahydrofuran with 0.06 wt. % surfactant | 2.75 |

The results in table 5 demonstrate that when a small amount of surfactant is added to the solvent system, less solvent system is needed to dissolve the same amount of bleach catalyst. In other words, when the solvent system comprises a solvent and a small amount of surfactant, the solubility of the bleach catalyst is higher than in the solvent alone. This implies that less solvent system is needed during the granulation process.0

### Performance of Bleach Catalysts

### Kinetic of Tropaeolin-O Degradation

These tests were performed following the procedure described in US2012/0205581 A1. Here, Tropaeolin-O was used as sensor molecule to describe the oxidation kinetic of the different catalysts. The catalytic activity of a bleach catalyst is given by the photocatalytic degradation of tropaeolin-O under visible light irradiation.

The kinetic of tropaeolin-O degradation of each sample was determined by measuring the UV-Vis absorbance of the sample every 60 seconds for a total period of 20 minutes at 430 nm. This was done using a UV-Vis Evolution 220 FC-1135 spectrophotometer using a 2 mm mm quartz cuvette for the measurements at 40° C. and a 1 mm quartz cuvette for the measurements at 20° C.

### Samples Preparation for Tropaeolin-O Degradation at 40° C.

A base detergent system containing tropaeolin-O was prepared by adding to 1000 mL of demineralized at 40° C., the following ingredients:
2.92 g of Ariel Gel (as sold in the UK),
0.088 g of Tropaeolin-O (purity: ex. Sigma and used as received),
1.75 g of Sodium percarbonate (purity: 13.3 wt. % active oxygen),
0.10 g of bleach activator tetraacetylethylenediamine (TEAD, purity: 92 wt. % active)

Detergent systems to be tested for the determination of the tropaeolin-O degradation were based on the base detergent system shown above to which a specific bleach catalyst was added, so that the concentration of the bleach catalyst in the detergent system as 0.0144 mM.

TABLE 6
Detergent systems for tropaeolin-O degradation

| Detergent System | Ingredients |
|---|---|
| Base | 2.92 g of Ariel Gel as sold in the UK, |
| | 0.088 g of Tropaeolin-O, |
| | 1.75 g of Sodium percarbonate, |
| | 0.10 g of bleach activator |
| | tetraacetylethylenediamine (TEAD) |
| Detergent system A | Base system + bleach catalyst A |
| | (concentration 0.0144 mM) |
| Detergent system B | Base system + bleach catalyst B |
| | (concentration 0.0144 mM) |
| Detergent system C | Base system + bleach catalyst C |
| | (concentration 0.0144 mM) |
| Detergent system 1 | Base system + bleach catalyst 1 |
| | (concentration 0.0144 mM) |
| Detergent system 2 | Base system + bleach catalyst 2 |
| | (concentration 0.0144 mM) |

### Kinetic of Tropaeolin-O Degradation at 40° C.

The 430 nm absorption peak associated to the decomposition of the sensor molecule, i.e., tropaeolin-O, after addition of a detergent system was followed over time. Table 7 shows the absorbance of tropaeolin-O at different times at 430 nm. Absorbance in Table 7 is given in absorbance units (a.u.)

TABLE 7
UV-Vis absorption at 430 nm of the Tropaeolin-O decomposition
kinetics for the detergent systems in Table6 at 40° C.

| | | Absorbance (a.u.) | | |
|---|---|---|---|---|
| Detergent System | pH | t = 0 min | t = 10 min | t = 20 min |
| Base | 10.2 | 1.43 | 1.41 | 1.36 |
| Detergent system A | 10.2 | 1.43 | 0.92 | 0.56 |
| Detergent system B | 10.2 | 1.43 | 1.37 | 1.29 |
| Detergent system C | 10.2 | 1.43 | 1.41 | 1.37 |
| Detergent system 1 | 10.2 | 1.43 | 0.33 | 0.18 |
| Detergent system 2 | 10.2 | 1.43 | 0.60 | 0.22 |

Table 5 shows that detergent systems containing a bleach catalyst with a ClogP value lower than 2 and a partial charge density of the C═N bond of less than −0.190, clearly have a better performance than those detergent systems containing a bleach catalyst with a ClogP higher than 2 and a partial charge density of the C═N bond of more than −0.190. This is evidenced as the absorbance values of the Tropaeolin-O sensor for the first bleach catalysts are lower during time, therefore associated to a higher reactivity with time.

### Samples Preparation for Tropaeolin-O Degradation at 20° C.—Granules

A base detergent system containing tropaeolin-O was prepared by adding to 1000 mL of demineralized at 20° C., the following ingredients:
2.92 g of Ariel Gel (as sold in the UK),
0.088 g of Tropaeolin-O (purity: ex. Sigma and used as received),
1.75 g of Sodium percarbonate (purity: 13.3 wt. % active oxygen),
0.10 g of bleach activator tetraacetylethylenediamine (TEAD, purity: 92 wt. % active)

Detergent systems to be tested for the determination of the tropaeolin-O degradation were based on the base detergent system shown above to a bleach catalyst or granules were added, so that the concentration of the bleach catalyst in the detergent system as 0.0144 mM.

TABLE 8
Detergent systems for tropaeolin-O degradation

| Detergent System | Ingredients |
|---|---|
| Detergent system 1C | Base system + bleach catalyst |
| | 1 added as pure crystal |
| | (concentration 0.0144 mM) |
| Detergent system 1G | Base system + granule 1 without |
| | surfactant (pure catalyst |
| | concentration 0.0144 mM) |
| Detergent system 1GS | Base system + granule 1 with |
| | surfactant (pure catalyst |
| | concentration 0.0144 nM) |

### Kinetic of Tropaeolin-O Degradation at 20° C.

The 430 nm absorption peak associated to the decomposition of the sensor molecule, i.e., tropaeolin-O, after addition of a detergent system was followed over time. Table 9 shows the absorbance of tropaeolin-O at different times at 430 nm. Absorbance in Table 9 is given in absorbance units (a.u.)

TABLE 9
UV-Vis absorption at 430 nm of the Tropaeolin-O decomposition
kinetics for the detergent systems in Table 8 at 20° C.

| | | Absorbance (a.u.) | | |
|---|---|---|---|---|
| Detergent System | pH | t = 0 min | t = 10 min | t = 20 min |
| Detergent system 1C | 10.2 | 1.43 | 1.41 | 1.37 |
| Detergent system 1G | 10.2 | 1.43 | 0.33 | 0.18 |
| Detergent system 2GS | 10.2 | 1.43 | 0.60 | 0.22 |

Table 9 shows that detergent systems containing in which the bleach catalyst is added as a granule have a better performance than those detergent systems in which the bleach catalyst is added in crystal form. Furthermore, when the granule is prepared using a solvent comprising a surfactant, the performance is better than a granule prepared with a solvent system devoid of surfactant. This is evidenced as the absorbance values of the Tropaeolin-O sensor for the detergent system comprising the granule prepared using a solvent system comprising a surfactant are lower during time, therefore associated to a higher reactivity with time.

### Tergotometer Test

The bleaching performance for real fabric staining was performed in a Tergotometer (TRG 800i Copley FC-1134) using the following technical stains:
W-10-LI Red Wine
C-S-34 Curry
W-10J Tea
W-10SG Spaghetti Sauce with meat
C-S-67 Mustard
C-S-15 Blueberry Juice

To carry out the tergotometer tests the detergent systems of Table 10 were used.

TABLE 10
Detergent systems for tergotometer test

| Detergent System | Ingredients |
|---|---|
| Ter-Base | 2.92 g of Ariel Gel (as sold in the UK), |
| | 1.75 g of Sodium percarbonate |
| | (13.3 wt. % active oxygen), |
| | 0.10 g of bleach activator |
| | tetraacetylethylenediamine |
| | (TEAD, purity: 92 wt % active) |
| Ter-Det. System A | Base system + bleach catalyst A |
| | (concentration 0.0144 mM) |
| Ter-Det. System B | Base system + bleach catalyst B |
| | (concentration 0.0144 mM) |
| Ter-Det. System C | Base system + bleach catalyst C |
| | (concentration 0.0144 mM) |
| Ter-Det. System 1 | Base system + bleach catalyst 1 |
| | (concentration 0.0144 mM) |
| Ter-Det. System 2 | Base system + bleach catalyst 2 |
| | (concentration 0.0144 mM) |

The tergotometer tests were carry out by dissolving the detergent system of table 10 to be tested in 1 litre of water. After approximately 1 minute the above-mentioned technical stains were added to the washing liquor. Four replicates for each technical stain were carried out, for a total of 24 experiments. Each test was performed at 20° C.±1 and at 40° C.±1, this to show the stain removal properties of each detergent system at two temperatures.

The technical stains were washed with the detergent system of Table 10 to be tested for approximately 30 minutes, in other words, each test lasted 30 minutes. After the technical stains were washed, these were allowed to dry overnight under a lotting paper covering and then ironed.

Finally, the stain removal performance of each detergent system was determined using a Datacolor 650-05 spectrophotometer by measuring the Y-value of the technical stain after washing. A higher Y-value means a better stain removal. The results of the tergotometer tests are shown in table 7; these results are provided as the average of the Y-values for bleachable stains at 20° C. and at 40° C. It is worth mentioning that the above-mentioned technical stains are bleachable stains.

TABLE 11
Tergotometer test results using the detergent systems in table
10. Values are given as Y-value average of all replicates.

| | pH tested | Y-value | Y-value |
|---|---|---|---|
| Detergent system | solution | at 20° C. | at 40° C. |
| Ter-Base | 10.2 | 67.6 | 76.1 |
| Ter-Det. System A | 10.2 | 68.8 | 78.1 |
| Ter-Det. System B | 10.2 | 67.7 | 76.5 |
| Ter-Det. System C | 10.2 | 67.8 | 76.5 |
| Ter-Det. System 1 | 10.2 | 68.4 | 77.9 |
| Ter-Det. System 2 | 10.2 | 71.8 | 78.8 |

As can be seen in Table 11, the detergent systems comprising the bleach catalysts according to the invention are particularly effective at removing stains at temperatures not only 40° C. but also at temperatures lower than 20° C.

### Washing Machine Test

The bleaching performance for real fabric staining was performed inside a Bosch washing machine (WAN282681T), using a “Mix 20° C.” washing protocol, with an estimated total length of washing cycle of about 60 min. The washing machine was loaded with 3.5 kg of ballast to mimic real usage conditions, and four external replicates were performed for each laundry detergent composition. The detergent systems in Table 12 were tested.

TABLE 12
Detergent systems for washing machine tests

| Detergent system | Ingredients |
|---|---|
| Det-WM-0 | 60 g commercial detergent |
| | 35 g laundry bleach composition of table 9 |
| Det-WM-A | 60 g of commercial detergent |
| | 35 g of laundry bleach composition of |
| | table 9 with a 0.014 mM |
| | concentration of bleach catalyst A. |
| Det-WM-1 | 60 g of commercial detergent |
| | 35 g of laundry bleach composition of |
| | table 9 with a 0.014 mM |
| | concentration of bleach catalyst 1. |
| Det-WM-2 | 60 g of commercial detergent |
| | 35 g of laundry bleach composition of |
| | table 9 with a 0.014 mM |
| | concentration of bleach catalyst 2. |

TABLE 13
Laundry bleach composition for use in washing machine tests

| Ingredient | Wt. % |
|---|---|
| Sodium Percarbonate (13.3 wt % active | 30-45 |
| oxygen) | |
| TAED (92 wt % active) | 2-5 |
| Bleach catalyst | 0.01-0.50 |
| Fillers | 45-60 |
| Surfactants | 0.5-5.0 |
| Enzymes | 0.5-2.0 |
| Dye transfer inhibitor | 0.1-1.0 |
| Additional ingredients | 0.5-2.0 |

In table 9:
The fillers are a combination of sodium carbonate, sodium sulphate, and zeolite,
The enzymes are protease, mannanase, lipase, amylase, cellulase, or combinations thereof,
The additional ingredients are fragrances, optical brighteners, or combinations thereof.

Technical stains were washed in the washing machine using the detergent system of Table 12. Stain removal was determined using a Datacolor 650 spectrophotometer and by measuring the Y-value of the standard soil after washing. A higher Y-value represents a better stain removal. The results are shown in table 14.

TABLE 14
Stain removal performance of detergent systems in table 12 in a washing machine.

| | | | Y-values-Detergent system Det-WM- | | | |
|---|---|---|---|---|---|---|
| | Standard soil | Description | 0 | A | 1 | 2 |
| Bleachable | C-BC-02 | coffee | 65.71 | 70.01 | 73.59 | 72.80 |
| | C-S-08 | grass | 80.14 | 79.51 | 80.81 | 80.52 |
| | C-S-12 | blackcurrant juice | 60.93 | 64.91 | 67.70 | 66.32 |
| | C-S-15 | blueberry juice | 67.96 | 70.99 | 74.23 | 73.46 |
| | C-S-67 | mustard | 81.73 | 82.90 | 84.64 | 83.44 |
| | E-164 | grass | 68.78 | 69.81 | 72.68 | 71.72 |
| | W-10J | tea | 71.45 | 72.57 | 75.04 | 73.48 |
| | W-10K | coffee | 83.60 | 85.86 | 87.61 | 87.60 |
| | W-10LI | wine | 69.54 | 69.45 | 73.20 | 72.57 |
| | W-10SG | spaghetti sauce with meat | 58.25 | 58.17 | 58.74 | 59.91 |
| | W-10U | curry | 84.63 | 85.97 | 88.04 | 88.19 |
| | WE5RWWKC | Red wine | 70.14 | 73.39 | 76.66 | 77.49 |
| | | Average | 71.91 | 73.63 | 76.08 | 75.62 |
| Enzymatic | C-S-06 | salad dressing/pigment | 62.43 | 63.21 | 61.84 | 60.67 |
| | C-S-27 | potato starch/colorant | 66.52 | 67.06 | 64.23 | 63.87 |
| | C-S-406 | Balsamic Dressing | 53.15 | 52.97 | 51.85 | 49.95 |
| | C-S-68 | chocolate ice cream | 69.22 | 69.72 | 72.53 | 71.15 |
| | E-111 | blood | 71.28 | 78.87 | 81.68 | 77.68 |
| | W-10Z | chocolate | 66.23 | 69.98 | 69.73 | 70.04 |
| | | Average | 64.80 | 66.97 | 66.98 | 65.56 |
| Detergency | C-S-17 | make up | 53.99 | 58.59 | 58.69 | 57.49 |
| | W-10D | skin grease/pigment | 58.81 | 65.34 | 64.01 | 59.58 |
| | W-10GM | motor oil used | 41.42 | 41.81 | 41.89 | 41.53 |
| | W-10MU | make up | 59.17 | 62.51 | 61.98 | 62.76 |
| | W-10RM | Soot mineral oil, IEC | 32.33 | 36.06 | 35.71 | 34.09 |
| | W-10V | soy sauce | 87.16 | 87.96 | 88.81 | 88.63 |
| | | Average | 55.48 | 58.71 | 58.52 | 57.34 |

(Each value in table 20 is the average of four measurements)

The results shown in Table 14 clearly indicate that the detergent systems comprising the bleaching agent according to the invention have a superior cleaning performance, in particular when considering the removal of bleachable stains.

Hence one or more objects of the present invention are achieved by the present which is further elucidated in the appended claims.

1. A bleach catalyst having formula (1): 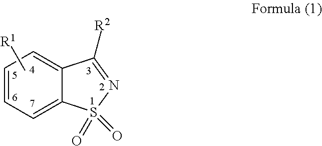
wherein:
R¹ is H, halogen, hydroxy, amino, nitro, cyano, carboxy, alkyl, C₁-C₁₀-alkoxy, C₁-C₁₀-alkylamino, phenyl, substituted phenyl, or a heterocyclic ring;
R² is an organic group comprising a carbon atom that is bonded to the third carbon of the bleach catalyst,
the partial charge density of the C═N bond of the bleach catalyst is less than −0.180, and
the ClogP of the bleach catalyst is lower than 2.

2. The bleach catalyst according to claim 1, wherein the ClogP of the bleach catalyst is lower than 1.8.

3. The bleach catalyst according to claim 1, wherein the partial charge density of the C═N bond is less than −0.190.

4. The bleach catalyst according to claim 1, wherein R¹ is H, hydroxy, or C₁-C₁₀-alkoxy.

5. The bleach catalyst according to claim 1, wherein R² is alkyl, heteroalkyl, alkenyl, alkynyl, aryl, substituted aryl, aryl with multiple or fused rings in which at least one ring is aromatic, heteroaryl, or heterocyclic ring.

6. The bleach catalyst of claim 1, wherein R² is an organic group in which the carbon atom that is bonded to the third carbon of the bleach catalyst is not bonded to any hydrogen atom.

7. The bleach catalyst according to claim 1, wherein R² is:
a branched alkyl or heteroalkyl having 4 to 16 carbon atoms;
a branched or cyclic alkenyl having 3 to 16 carbon atoms;
a heterocyclic alkenyl;
a cycloalkyl-, cycloalkenyl-, cycloalkynyl-, or aryl-substituted linear or branched alkenyl having 2 to 16 carbon atoms in the alkenyl chain;
a linear alkynyl having 2 to 16 carbon atoms;
a branched alkynyl having 5 to 16 carbon atoms;
a cycloalkyl-, cycloalkenyl-, cycloalkynyl-, or aryl-substituted linear or branched alkynyl having 2 to 16 carbon atoms in the alkynyl chain;
a substituted aryl;
an aryl with multiple or fused rings in which at least one ring is aromatic; or
a heteroaryl.

8. The bleach catalyst according to claim 7, wherein R² is:
a branched alkyl or heteroalkyl having 4 to 16 carbon atoms, or
a cycloalkyl-, cycloalkenyl-, cycloalkynyl-, or aryl-substituted linear or branched alkyl having 2 to 16 carbon atoms in the alkyl chain;
then the carbon in R² that is bonded to the third carbon atom of the bleach catalyst having formula (1) is a quaternary carbon atom.

9. The bleach catalyst according to claim 7, wherein R² is:
a heterocyclic alkenyl,
a substituted aryl,
an aryl with multiple or fused rings in which at least one ring is aromatic, or
a heteroaryl.

10. The bleach catalyst according to claim 9, wherein R² is a heterocyclic alkenyl or a heteroaryl.

11. The bleach catalyst according to claim 10, wherein R² is pyridyl, quinolyl, isoquinolinyl, acridinyl, pyrazinyl, quinoxalinyl, imidazolyl, benzimidazolyl, purinyl, pyrazolyl, indazolyl, pyrimidinyl, quinazolinyl, pyridazinyl, or cinnolinyl.

12. The bleach catalyst according to claim 11, wherein R² is a pyridyl.

13. A granule comprising the bleach catalyst according to claim 1 and a carrier.

14. The granule according to claim 13, wherein the carrier is selected from the group consisting of sulphates, carbonates, sesquicarbonates, silicates, aluminosilicates, zeolites, precipitated silicas, fumed silicas, and combinations thereof.

15. The granule according to claim 13, wherein the granule comprises between 0.1 wt. % and 40 wt. % of the bleach catalyst, based on the total weight of the granule.

16. The granule according to claim 13, wherein the granule further comprises at least one surfactant.

17. The granule according to claim 13, wherein the granule further comprises a binder.

18. The granule according to claim 13, wherein the granule has a particle size of between 200 μm and 2000 μm.

19. A method of preparing the granule according to claim 13, wherein the method comprises the steps of:
a) Preparing a solution by dissolving the bleach catalyst in a solvent system,
b) Adding the solution prepared in step a) to the carrier,
c) Allowing the solvent system to evaporate partially or completely,
wherein the solvent system has a dielectric constant below 80, a vapour pressure above 5 kPa and a boiling point below 100° C.

20. The method according to claim 19, wherein the solvent system comprises one or more solvents selected from the group consisting of water, methanol, ethanol, propylene carbonate, acetone, acetonylacetone, diacetone alcohol, ethyl acetate, 2-propanol, ethylene glycol, propylene glycol, glycerine, diethylene glycol, dipropylene glycol monomethyl ether and dimethylformamide.

21. The method according to claim 20, wherein the solvent system further comprises at least one surfactant.

22. A cleaning composition comprising the bleach catalyst according to claim 1 and/or the granule according to claim 13, at least one bleaching agent and at least one bleach activator.

23. The cleaning composition according to claim 22, wherein the at least one bleaching agent and/or the at least one bleach activator is/are partially or totally contained in the granule.

24. (canceled)

25. The cleaning composition according to claim 22, wherein the composition comprises between 0.001 wt. % and 10 wt. % of the bleach catalyst, based on the total weight of the composition.

26. The cleaning composition according to claim 22, wherein the at least one bleaching agent comprises at least one source of active oxygen selected from the group consisting of an inorganic peroxide, an organic peracid, hydrogen peroxide, and combinations thereof.

27. The cleaning composition according to claim 22, wherein the at least one bleach activator is selected from the group consisting of tetraacetylethylenediamine (TAED), acetylated triazine derivatives, acetylated glycoluriles, acylimides, acetylated glucose, acetylated glycerol, acetylated phenolsulfonates, acetylated phenol carbonic acids, sodium nonanoyloxybenzenesulfonate (NOBS), 4-decanoyloxybenzoic Acid (DOBA), carbonic acid anhydrides, acetylated sugar derivatives, N-acetylated lactams, and combinations thereof.

28. A method of using the cleaning composition according claim 22 for the removal of stains and/or for the removal of malodour from a garment, the method comprising: washing the garment with the cleaning composition of claim 22, wherein the stains comprise a polyphenolic compound, are turmeric stains, and/or are curry stains.

29. (canceled)

30. A method of washing clothes, wherein the method comprises providing the composition according to claim 22 to a laundry washing machine and performing the washing:
at a temperature lower than 60° C.; and
for a time of less than 120 minutes.
